(19) United States
(12) Patent Application Publication
(10) Pub. No.: US 2006/0035289 A1
(43) Pub. Date: Feb. 16, 2006

(54) Affinity fluorescent proteins and uses thereof

(75) Inventors: Paul T. Matsudaira, Wayland, MA (US); Daniel J. Ehrlich, Lexington, MA (US); Qiuhui Zhong, Lexington, MA (US); Yelena Freyzon, Chestnut Hill, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Whitehead Institute for Biomedical Research, CAMBRIDGE, MA (US)

(21) Appl. No.: 10/978,788

(22) Filed: Nov. 1, 2004

Related U.S. Application Data
(63) Continuation of application No. 09/627,383, filed on Jul. 28, 2000, now abandoned.
(60) Provisional application No. 60/146,438, filed on Jul. 29, 1999.

(51) Int. Cl. G01N 33/567; C07K 14/435

(52) U.S. Cl. 435/7.21; 530/350

(57) ABSTRACT

The present invention is related to an affinity fluorescent protein (aFP) comprising a modified fluorescent protein or molecule which comprises a heterologous amino acid sequence, thereby introducing a ligand-activated protein binding site, wherein the modified fluorescent protein displays an altered spectral property when the binding site is engaged with ligand relative to the spectral property displayed when the binding site is not engaged by ligand. The present invention also relates to an aFP expression cassette comprising a modified fluorescent protein nucleic acid sequence operatively linked to expression control sequences, wherein the modified fluorescent protein sequence comprises a recombinant peptide which comprises restriction endonuclease sites; and a host cell, comprising a recombinant nucleic acid molecule which comprises expression control sequences operatively linked to nucleotide sequence encoding an aFP, wherein said aFP comprises modified GFP molecule which comprises a mutated GFP molecule and a heterologous amino acid sequence which functions as a ligand-activated protein binding site, wherein the aFP an altered spectral property when the binding site is engaged with ligand relative to the spectral property displayed when the binding site is not engaged by ligand. The present invention also relates to a method of detecting the presence of a target ligand in a mixture of macromolecules. Also encompassed by the present invention is a method of a method of detecting the occurrence of a target ligand in a cell (e.g., a macrophage, a yeast cell).

### RELATED APPLICATION(S)

This application is a continuation of U.S. application Ser. No. 09/627,383, filed Jul. 28, 2000, which claims the benefit of U.S. Application No. 60/146,438, filed Jul. 29, 1999. This application is related to U.S. Application No. 60/061,801, entitled, “Affinity Fluorescent Proteins and Uses Therefor,” filed on Oct. 14, 1997. The entire teachings of the above application(s) are incorporated herein by reference

### GOVERNMENT SUPPORT

The invention was supported, in whole or in part, by a grant F30602-97-2-0272 from DARPA. The Government has certain rights in the invention.

### BACKGROUND OF THE INVENTION

Many methods are available for detecting, quantifying, locating and purifying proteins and other molecules of interest. Additional methods and reagents, particularly those which are more specific, faster to use and less expensive than those presently available, would be desirable.

### SUMMARY OF THE INVENTION

Described herein are ligand-activated fluorescent biosensor proteins referred to as affinity fluorescent proteins (aFP). The aFP of the present invention embody a new class of proteins derived from an Aequorea-related protein (e.g., green fluorescent protein (GFP)) which comprise a heterologous amino acid sequence, which functions as a ligand (e.g., an enzyme, protease inhibitor or antibody) binding site, introduced into select surface loops of the Aequorea-related protein. The aFP described herein can noncovalently bind a variety of molecules (e.g., natural, synthetic, biological, non-biological, organic, inorganic, protein, non-protein small or large) and is capable of functioning both as molecular recognition moieties and as molecular biosensors which are capable of sensing and reporting the interaction of a binding site with its cognate ligand. This is done, for example, by inserting a specially designed loop between one or both ends of the chromophore helix and the surrounding beta-barrel. Such modification(s) allow non-covalent binding to be coupled with an instantaneous change in fluorescence (intensity and/or spectrum). The affinity and specificity of binding can be further tailored by additional mutation of the same loop or to mutations in surrounding loops. Furthermore, additional groups to the N or C terminus of the protein can permit non-covalent or covalent binding to an inert surface.

More specifically, the present invention is related to an affinity fluorescent protein (aFP) comprising a modified fluorescent protein or molecule, such as a modified GFP molecule, which comprises a heterologous amino acid sequence (one or more), thereby introducing a ligand-activated protein binding site, wherein the modified fluorescent protein displays an altered spectral property (e.g., an altered absorption spectra, an altered excitation spectra, an altered emission spectra and any combination thereof) when the binding site is engaged with ligand relative to the spectral property displayed when the binding site is not engaged by ligand. In the aFP of the present invention, the fluorescent protein can be mutated (e.g., so that the fluorescence of the aFP is stabilized). In a particular embodiment, the aFP of the present invention comprises a mutated GFP molecule in which serine at position 147 is replaced with a proline (a Ser147Pro (S147P) substitution). In another embodiment, the modified fluorescent protein of the aFP comprises one or more protein binding sites introduced at a single site in tandem or introduced at distinct sites as separate heterologous sequences. For example, in one embodiment, the modified GFP molecule in the aFP can comprise protein binding sites introduced into a loop present on the surface of the GFP molecule wherein the presence of the heterologous amino acid sequences does not alter the spectral properties of the GFP. In particular embodiments, the modified GFP molecule of the aFP can comprise at least one heterologous amino acid sequence (e.g., a protein binding site) introduced into the N-terminus, between Gln157 and Lys158, between positions Glu172 and Asp173, the C-terminus and any combination thereof.

The present invention also relates to an aFP expression cassette (vector) comprising a modified fluorescent protein nucleic acid sequence operatively linked to expression control sequences, wherein the modified fluorescent protein sequence comprises a recombinant peptide which comprises restriction endonuclease sites. In one embodiment, the present invention relates to an aFP expression cassette comprising a modified GFP nucleic acid sequence operatively linked to expression control sequences, wherein the modified GFP sequence comprises a recombinant peptide which comprises restriction endonuclease sites introduced at a location of the GFP molecule selected from the group consisting of between Gln157 and Lys158, between Glu172 and Asp173 and both of the aforementioned locations. In one embodiment, the aFP expression cassette comprises the hexapeptide LEPRAS (SEQ ID NO: 1). In a particular embodiment, the GFP molecule is mutated.

The present invention also relates to a host cell, comprising a recombinant nucleic acid molecule which comprises expression control sequences operatively linked to nucleotide sequence encoding an aFP, wherein said aFP comprises a heterologous amino acid sequence which functions as a ligand-activated protein binding site, wherein the aFP an altered spectral property when the binding site is engaged with ligand relative to the spectral property displayed when the binding site is not engaged by ligand. In one embodiment, the aFP comprises a modified GFP molecule, and in a particular embodiment, the GFP molecule is mutated (e.g., S147P). In the embodiment in which the fluorescent protein is GFP, the heterologous amino acid sequence can be introduced at a location selected from the group consisting of between Gln157 and Lys158, between Glu172 and Asp173 and both of the aforementioned locations.

The present invention also relates to a method of detecting the presence of a target ligand in a mixture of macromolecules. In the method, a sample to be evaluated for the presence of a target ligand molecule (test sample) is prepared and contacted with an aFP which comprises a binding site for the target ligand. The aFP is excited with light, and the fluorescent property that differs as a result of ligand activation of the aFP is measured (e.g., using a solid phase support such as nitrocellulose). The fluorescent property that differs as a result of ligand activation is selected from the group of properties consisting of amplitude of the excitation, absorption or emission spectra and shape of the any of the aforementioned spectras. In one embodiment, the aFP comprises a modified fluorescent protein or molecule, such as a modified GFP molecule, which comprises a heterologous amino acid sequence, thereby introducing a ligand-activated protein binding site, wherein the modified fluorescent protein displays an altered spectral property when the binding site is engaged with ligand relative to the spectral property displayed when the binding site is not engaged by ligand. In a particular embodiment, the GFP molecule is mutated.

Also encompassed by the present invention is a method of a method of detecting the occurrence of a target ligand in a cell (e.g., a macrophage, a yeast cell). In this method, an aFP which comprises a binding site for the target ligand is introduced into the cell, and the aFP present in the cell is excited with light. A pattern of fluorescence in the cell is then detected and compared to the pattern of fluorescence in a control cell, wherein the pattern of fluorescence determines the occurrence of the target ligand in the cell. In one embodiment, the aFP comprises a modified fluorescent protein or molecule, such as a modified GFP molecule, which comprises a heterologous amino acid sequence, thereby introducing a ligand-activated protein binding site, wherein the modified fluorescent protein displays an altered spectral property when the binding site is engaged with ligand relative to the spectral property displayed when the binding site is not engaged by ligand. In a particular embodiment, the GFP molecule is mutated.

The ligand-activated fluorescent biosensors described herein are useful to detect and monitor a range of in vitro and in vivo biological activities which include, but are not limited to, specific molecular processes in cells (e.g., membrane processes, intracellular signaling processes), cellular physiology, and the detection, quantification and/or purification a target ligand (e.g., a protease) from a wide variety of samples (e.g., cell lysates and tissue sections). For example, an aFP described herein can be used to detect (e.g., sense and report) the presence of a single target ligand in a complex mixture of macromolecules present in a cellular lysate, a mixture of macromolecules and/or a target cell. Thus, the disclosed biosensor proteins can be used, for example, as a substitute for reporter-molecule labeled monoclonal or polyclonal antibodies.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1A is a schematic representation of the concept and goal of producing an affinity fluorescent protein. FIG. 1B is a schematic representation of potential regions of GFP which can potentially accommodate guest loops comprising heterologous ligand binding sites. The white numbered bars signify 11 antiparallel B sheets and the shaded bar represents a single central α helix. The position of the chromophore (Ser 65-Tyr 66-Gly67) (SEQ ID NO: 2) is indicated on the α helix.

FIGS. 2A-2E depict the fluorescence excitation spectra of various affinity fluorescent proteins comprising the haemagglutinin epitope in the presence and absence of the anti-haemagglutinin antibody 12CA5. The concentrations of HA2 mutants, anti-HA, and anti-ACT (monoclonal antibody against anti-chymotrypsin) were 0.3 mg/ml, 3.4 mg/ml, 3.4 mg/ml respectively. The spectra were collected at fixed emission wavelength of 550 nm. The red lines represented complexes. The green lines represented mutants alone. The blue lines represented mutants plus an nonspecific antibody as negative control.

FIGS. 3A-3E depict the fluorescence emission spectra of various affinity fluorescent proteins comprising the haemagglutinin epitope in the presence and absence of the ant-haemagglutinin antibody 12CA5. The concentrations of HA2 mutants, anti-HA, and anti-ACT (monoclonal antibody against anti-chymotrypsin) were 0.3 mg/ml, 3.4 mg/ml, 3.4 mg/ml respectively. The spectra were collected at fixed excitation wavelength of 395 nm. The red lines represented complexes. The green lines represented mutants alone. The blue lines represented mutants plus a nonspecific antibody as negative control.

FIGS. 4A-4E depict the fluorescence absorption spectra of various affinity fluorescent proteins comprising the haemagglutinin epitope in the presence and absence of the ant-haemagglutinin antibody 12CA5. The concentrations of HA2 mutants, anti-HA, and anti-ACT (monoclonal antibody against anti-chymotrypsin) were 0.3 mg/ml, 3.4 mg/ml, 3.4 mg/ml respectively. The red lines represented complexes. The green lines represented mutants alone. The blue lines represented mutants plus a nonspecific antibody as negative control.

FIG. 5 depicts the fluorescence excitation and emission spectra of an affinity fluorescent protein (aFP) comprising the hemagglutinin epitope in the presence and absence of the anti-hemagglutinin antibody 12 CAS.

FIGS. 6A and 6B comprise schematic representation of the various affinity fluorescent proteins comprising the haemagglutinin epitope YPYDVPDYA (SEQ ID NO: 3) (HA residues 98-106) described herein. The shaded boxes represent the haemagglutinin epitope.

FIG. 7 is a schematic representation of ligand-affinity fluorescence biosensor derived from the GFP. The aFP is composed of one or more binding sequences presented on the surface loops. The binding to the ligand likely results in different fluorescence properties, such as enhanced, quenched or shifted fluorescence.

FIGS. 8A-8C depicts the absorption spectra of HA2 mutants and complexes with anti-HA. The concentrations of HA2 mutants, anti-HA and anti-ACT (monoclonal antibody against antichymotrypsin) were 0.3 mg/ml, 3.4 mg/ml and 3.4 mg/ml, respectively. The red lines represent complexes. The green lines represent mutants alone. The blue lines represent mutants plus a nonspecific antibody as a negative control.

FIGS. 9A-9E depicts excitation spectra of HA2 mutants and complexes with anti-HA. The concentrations of HA2 mutants, anti-HA, anti-ACT and bovine serum albumin (BSA) were 0.025 mg/ml, 0.2 mg/ml, 0.2 mg/ml and 0.2 mg/ml, respectively. The spectra were collected at fixed emission wavelength of 550 nm. The red lines represent complexes. The green lines represent mutants alone. The purple lines represent mutants plus a nonspecific antibody as a negative control. The blue line represents the mutants plus BSA.

FIGS. 10A-10E depicts emission spectra of HA2 and complexes with anti-HA. The concentrations of HA2 mutants, anti-HA, anti-ACT and BSA were at 0.025 mg/ml, 0.2 mg/ml, 0.2 mg/ml and 0.2 mg/ml, respectively. The spectra were collected at fixed excitation wavelength of 395 nm. The red lines represent complexes. The green lines represent mutants alone. The purple lines represent mutants plus a nonspecific antibody as a negative control. The blue lines represent mutants plus BSA.

FIG. 11 depicts the emission spectra for the 172HA2 on the nitrocellulose membrane after wash. The thin line represents 172HA2 alone, the thick line represents 172HA2/anti-HA complex and the dot line represents 172HA2 with BSA.

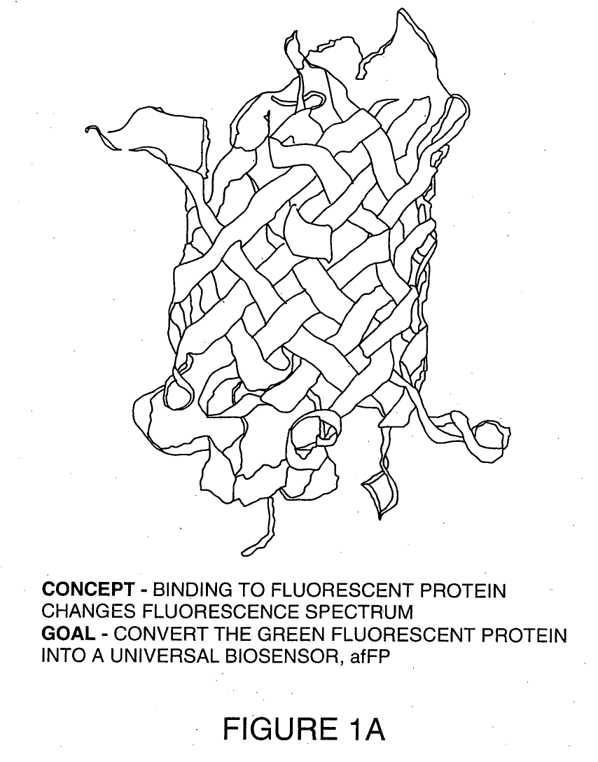

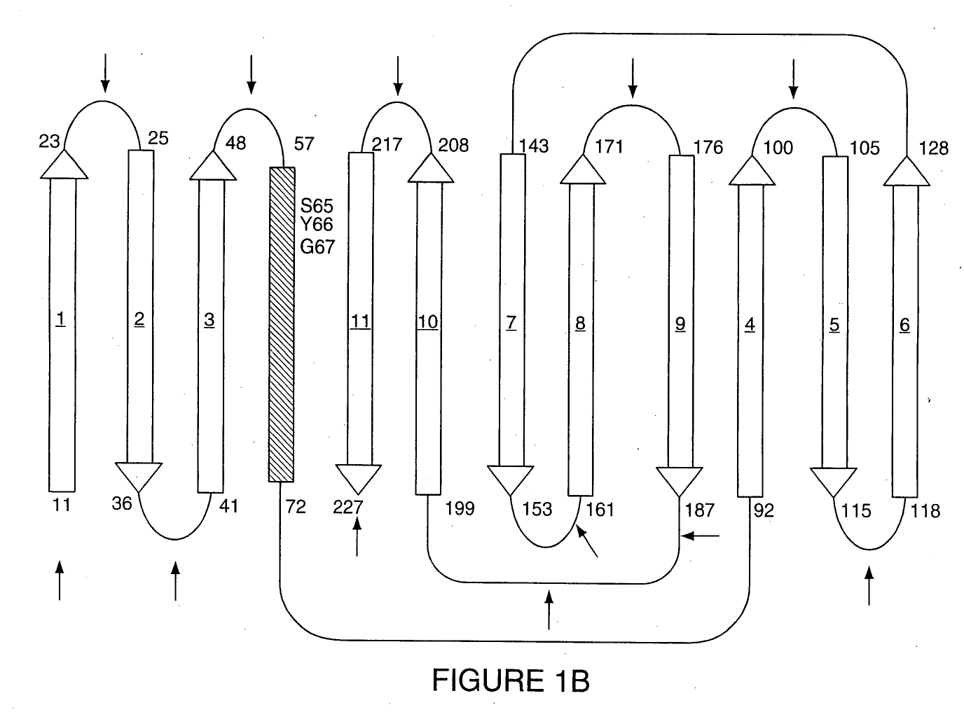

### DETAILED DESCRIPTION OF THE INVENTION

By inserting peptide binding sequences into the surface loops of a fluorescent protein, affinity fluorescent protein (aFP) sensors which are able to report binding of a specific ligand based on the enhancement of fluorescence intensity have been developed. As a model system, the epitope of haemagglutinin (HA) tag that is recognized by the monoclonal antibody (anti-HA or 12CA5) was chosen. A single HA tag or two tandem HA tags were inserted into three locations on the green fluorescent protein (GFP) surface loops. Excitation and emission spectra of aFP/anti-HA complexes were enhanced upon binding to anti-HA antibody in solution for the aFPs with high affinities. To increase the sensitivity and simplify the detection, a solid surface binding assay based on the enhancement of fluorescence intensity, which is able to detect weak binding between the low affinity aFPs and the anti-HA antibody, was developed. The aFPs described herein provide for a rapid and efficient method for detecting protein-protein interactions.

The aFP of the present invention can be, for example, a modified Green Fluorescent Proteins (GFP) which exhibit specific affinity to a target molecule (e.g., ligand) and are able to sense and report the interaction of the binding site and the target ligand. The reporter, or indicator, function of the aFP is attributed to the alteration of one of the spectral properties (e.g., adsorption, excitation or emission) of the fluorescent protein. The engagement (e.g., non-covalent binding) of the heterologous amino acid sequence (e.g., binding site) present in an aFP described herein is accompanied by a rapid, typically instantaneous, and measurable alteration in a spectral property of the modified GFP's fluorescence. For example, the altered fluorescence characteristics associated with ligand binding can include altered excitation and emission maxima or altered adsorption or excitation spectra.

As described herein, aFPs were produced by introducing heterologous amino acid sequences (e.g., peptides or epitopes) encoding particular ligand binding sites, into select regions of the wild type molecule which correspond to loops present on the surface of a fluorescent protein. For example, a single ligand binding site was introduced between Gln157 and Lys158 (e.g., 157HA) or Glu172 and Asp173 (e.g., 172HA2) of the GFP molecule thereby producing a modified GFP suitable for use as ligand-activated fluorescent biosensor proteins. GFP is a 238 amino acid protein and is composed of 11 beta-strand barrel and five surface loops at each end of the barrel wrapping the cyclic chromophore (Thr65-Tyr66-Gly67) residing on a distorted alpha-helix located inside of the barrel (Yang et al.). Any fluorescent protein having fluorescent insensitive sites into which can be introduced a (one or more) ligand-activated protein binding site, thereby producing a modified fluorescent protein, and wherein the modified fluorescent protein displays an altered spectral property when the binding site is engaged with ligand can be used in the present invention. Examples of fluorescent proteins which can be used in the compositions and methods of the present invention include, for example, GFP, yellow fluorescent protein (YFP) (GFP mutant: S65G; V68L; Q69K; S72A; T203Y), cyan fluorescent protein (CFP) (GFP mutatn: F64L; S65T; T66W; N146I; M153T), blue fluorescent protein (BFP) (GFP mutant: F64L; S65T; Y66H; Y145F). Other examples include fluorescent proteins recently cloned by amplification of cDNAs from nonbioluminescent Anthoza species (corals of the Indian and Pacific Oceans) which include yellow and red-orange emitters with 26% to 30% sequence identity to GFP and several conserved features of GFP structure including the 11 stranded and beta-strand (B-can) fold, Arg96 and Glu222 (Matz, M. V., et al., Nat. Biotechnol., 17(10):969-973 (1999).

To facilitate the identification of fluorescence insensitive sites which can accommodate the presence of a binding site and/or the production aFPs, an affinity fluorescent protein cassette can be created by introducing a small synthetic test peptide comprising one or more appropriate restriction enzymes sites at candidate locations in the sequence of a fluorescent protein such as GFP. After identifying regions (e.g., guest loops) of the fluorescent protein which can tolerate the introduction of the test peptide without a lose of flourescent intensity, the restriction sites can be used for the introduction of heterologous amino acid sequences, or non-protein moieties which embody the desired binding site. For example, the hexapeptide LEPRAS (SEQ ID NO: 1) which contains three restriction enzyme sites (XhoI-AvrII-NheI) was useful for identifying flourescent insensitive sites in the GFP molecule. Alternatively, other test peptides can be designed which exhibit characteristics such as hydrophobicity and charge in common with either the native loop or with the heterologous amino acid sequence or moiety selected for introduction into the fluorescent protein. Affinity and specificity of binding of an aFP of the present invention can be further tailored by additional modification of the same loop, for example, by introducing two or more binding sites (e.g., linear or cyclic peptides) in tandem at a single location, or by introducing the same binding site at distinct locations. For example, two binding sites can be introduced at the position between Gln157 and Lys158 (e.g., 157HA2 or 157HA2) or at the position between Glu172 and Asp173 (e.g., 172HA, 172HA2). Alternatively, a single copy of each binding site can be introduced at two or more distinct sites (e.g., 157HA/172HA). Depending on the nature of the target ligand, affinity of the binding of an aFP of the present invention may also be enhanced by introducing an additional binding sites at either, or both, the N-terminus and C-terminus (e.g., 157/CHA) of the GFP molecule.

The magnitude of the spectral change, and thus the sensitivity of the biosensor, can be further tailored by introducing additional mutations, such as point mutations into the fluorescent protein amino acid sequence. For example, it has been reported that a mutation at position 147 from serine to proline facilitates protein folding, and thus the formation of the GFP chromophore (Ser65-Tyr66-Gly67). Alternatively, the modifications to introduce a heterologous amino acid sequence into a GFP protein described herein, can be introduced into a GFP mutant which has been genetically engineered to confer particular spectral properties to the starting protein. For example, U.S. Pat. No. 5,804,387 describes three GFP mutants suitable for use as starting proteins for the production of aFP described herein. GFPmut1 has a double substitution: F64L, S65T; GFPmut2 has a triple substitution: S65A, V68L, S72A; and GFPmut3 is characterized by the double substitution S65G, S72A. The commercial availability of cloning vectors comprising the nucleotide sequences encoding these various forms of GFP facilitate the design, production and expression of aFP. For example, the cloning vector pEGFP (Clontech Catalog #6077-12) encodes the GFPmut1 variant which produces a modified red-shifted variant of wild-type green fluorescent protein which has been optimized for brighter fluorescence and higher expression in mammalian cells.

In an alternative embodiment of the invention described herein, the aFP can be an Aequorea-related protein whose amino acid sequence has been modified to comprise heterologous binding sites at guest loop locations which result in the production of a protein which is not fluorescent in the absence of ligand engagement of the binding site, but upon ligand engagement would undergo a structural/conformation change which would allows for cyclization of the side chain and formation of the chromophore. In this embodiment of the invention, the chromophore would function as a true reporter of molecular interaction because fluorescence would be absent when the binding site was not engaged by ligand and present only when the ligand-activated binding site is engaged by a cognate ligand having specificity for the binding site.

The addition of groups to the amino- and or carboxyl-terminus of the aFP will permit the covalent or noncovalent binding of the aFP to an inert surface (e.g., surface of multiwell assay plates, beads and chips) thereby facilitating use of the biosensors in high-throughput drug discovery methods. Alternatively, the addition of certain functional groups as tags may also facilitate the purification of the aFP, for example, the introduction of a histidine tag would allow for a one-step purification method using a nickel binding column.

The aFPs, or protein sensors, of the present invention are multifunctional, they can purify, detect, quantify and locate a given ligand in a variety of environments, from a tissue section to an aqueous solution. An aFP is not only cheap and fast to produce, but its replacement of antibodies will increase the specificity, range and speed of immunoassays by orders of magnitude. Such aFPs have applications in medicine and biology but also in non-biological areas such as chemistry and engineering.

Accordingly, the present invention relates to a method of detecting the presence of a target ligand in a complex mixture of macromolecules. In the method, a sample to be evaluated for the presence of a target ligand molecule (test sample) is prepared and contacted with an aFP which comprises a binding site for the target ligand. The aFP is then excited with light at a wavelength which is appropriate for the particular fluorescent protein being used and can be determined empirically. For example, it is known that the appropriate excitation wavelength for GFP is from about 320 nm to about 500 nm, and in particular, can be about 395 nm and/or about 495 nm. An appropriate excitation wavelength for a red or yellow fluorescent protein can range as high as about 560 nm. The fluorescent property that differs as a result of ligand activation of the aFP is measured (e.g., using a solid phase support such as nitrocellulose). In one embodiment of the method, the fluorescent property that differs as a result of ligand activation is selected from the group of properties consisting of amplitude of the excitation, absorption or emission spectra and shape of the any of the aforementioned spectras. In another embodiment, the aFP comprises a modified fluorescent protein or molecule, such as a modified GFP molecule, which comprises a heterologous amino acid sequence, thereby introducing a ligand-activated protein binding site, wherein the modified fluorescent protein displays an altered spectral property when the binding site is engaged with ligand relative to the spectral property displayed when the binding site is not engaged by ligand. In a particular embodiment, the GFP is mutated.

The present invention also encompasses a method of a method of detecting the occurrence of a target ligand in a cell (e.g., a macrophage, a yeast cell). In this method, an aFP which comprises a binding site for the target ligand is introduced into the cell, and the aFP present in the cell is excited with light. A pattern of fluorescence in the cell is then detected and compared to the pattern of fluorescence in a control cell, wherein the pattern of fluorescence determines the occurrence of the target ligand in the cell. In one embodiment, the aFP comprises a modified fluorescent protein or molecule, such as a modified GFP molecule, which comprises a heterologous amino acid sequence, thereby introducing a ligand-activated protein binding site, wherein the modified fluorescent protein displays an altered spectral property when the binding site is engaged with ligand relative to the spectral property displayed when the binding site is not engaged by ligand. In a particular embodiment, the GFP is mutated.

The present invention also relates to a host cell, comprising a recombinant nucleic acid molecule which comprises expression control sequences operatively linked to nucleotide sequence encoding an aFP, wherein said aFP comprises a heterologous amino acid sequence which functions as a ligand-activated protein binding site, wherein the aFP an altered spectral property when the binding site is engaged with ligand relative to the spectral property displayed when the binding site is not engaged by ligand. In one embodiment, the aFP comprises a modified GFP molecule, and in a particular embodiment, the GFP molecule is mutated (e.g., S147P). In the embodiment in which the fluorescent protein is GFP, the heterologous amino acid sequence can be introduced at a location selected from the group consisting of between Gln157 and Lys158, between Glu172 and Asp173 and both of the aforementioned locations.

A variety of methods can be used to detect and/or measure the fluorescent property that differs as a result of ligand activation of the affinity fluorescent protein. For example, fluorescence can be measured using native electrophoresis, a spectrophotometer and/or a binding assay. The binding assay can be performed uinsg, for example, a solid support phase which can immobilize (bind) the aFP while retaining the function and structure of the aFP can be used. For example, nitrocellulose can be used as a solid support phase for use in detecting fluorescence of the aFPs.

In addition, fluorescence of the aFPs of the present invention can be detected using protein chip technology (e.g., NTA chip (Biacore)). aFPs constitute reagents for array analysis and will be the first demonstration of an array chip for proteins and cells. The procedure will significantly speed, simplify and reduce the costs of generating antibody-like reagents. The protein chip will be an array style device addressable by laser and detected by CCD.

Protein analysis generally falls into three categories: detection or identification, characterization and quantification. In basic science labs, the biggest need is for highly sensitive methods. Generally, the range in amounts needed for analysis varies greatly (10⁻⁹-10⁻¹⁸ moles) depending on the method. To detect a protein, the most sensitive methods include autoradiography of radioactive proteins, consumption of substrate or generation of product by enzymes, or reactivity with antibodies. Direct identification of a protein requires a limited amount (10-20 residues) of sequence or a highly specific antibody. Recent development of powerful time-of-flight mass spectrometers together with the compilation of sequences by the Genome Project will simplify identification by at least three orders of magnitude. Currently, mass spectrometers can analyze fmol-pmol quantities of sample, this limit is expected to reach atamol-fmol within three years. By far, the least sensitive methods are those that quantify the amount of protein. Standard laboratory assays are spectrophotometric tests that rely on the development of color by ng-ug quantities of sample. Radioimmunoassays using monoclonal antibodies quantify proteins at the pg level.

Other factors which influence the usefulness of an assay are speed, cost and accuracy, but unlike sensitivity the limits of these parameters have not been reached or defined. For instance, antibody based methods generally require 4-12 hours. One trend to improve assays is to reduce the volume to μl scales. With smaller volumes, less reagent and material is used and assays are performed more quickly. A benefit of volume reduction is that more samples can be processed by smaller instruments.

Small-scale protein analysis is a cumbersome process which requires metabolic labeling of cellular proteins, separation by 2D gel electrophoresis and autoradiographic detection of the radio-labeled proteins. A variation of this approach is to identify a particular protein by detecting the region of the gel that is bound by a specific antibody. These methods represent a general and a precise approach for analyzing a population of proteins. However, in most applications, proteins to be analyzed are known. Thus, analysis of a select population of proteins is diagnostic of a particular chip or a metabolic state of a cell. A protein “chip” for analysis of both proteins and cells can be developed. The chip can contain an array of custom engineered affinity fluorescent proteins which are specific for a set of proteins.

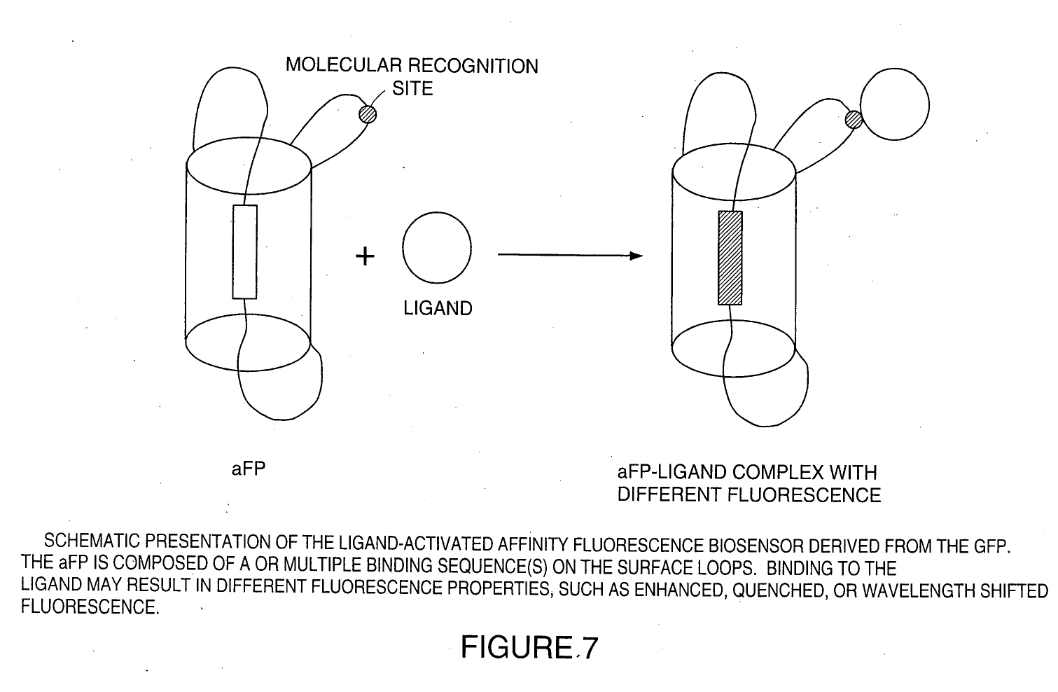

To combine the GFP's capability of displaying foreign peptide sequences and its autofluorescence as a reporter, a ligand-activated fluorescent protein (aFP) biosensor that is able to detect protein-protein interactions (FIG. 7) has been developed as described herein. The aFPs having molecular recognition site(s) on the surface loops can recognize targets, bind to them, and report the binding. The binding can be reported through enhanced, quenched, or wavelength shift fluorescence. The affinity fluorescent protein (aFP) sensors described herein not only bind to a target protein specifically, but also report the binding by increasing fluorescence intensity.

### EXAMPLE 1

### Creation of an Affinity Fluorescent Protein

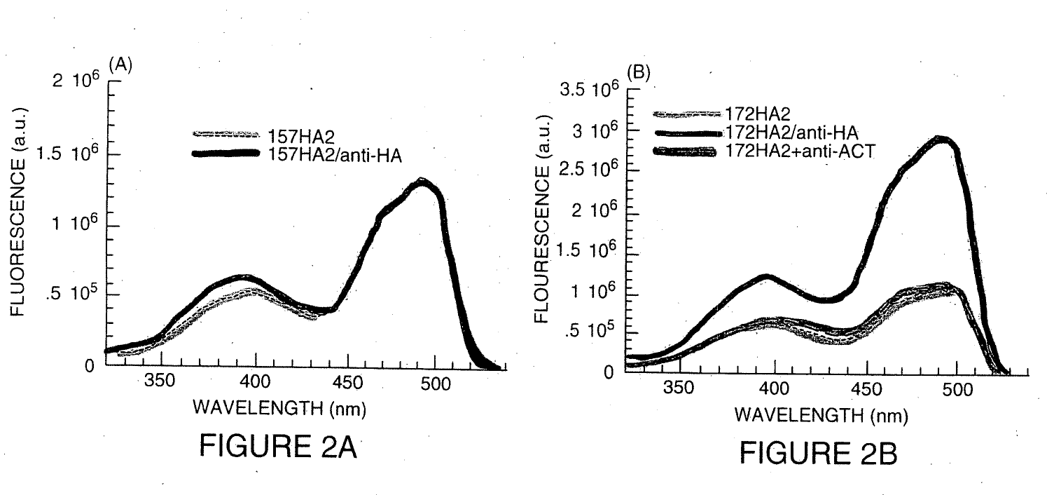

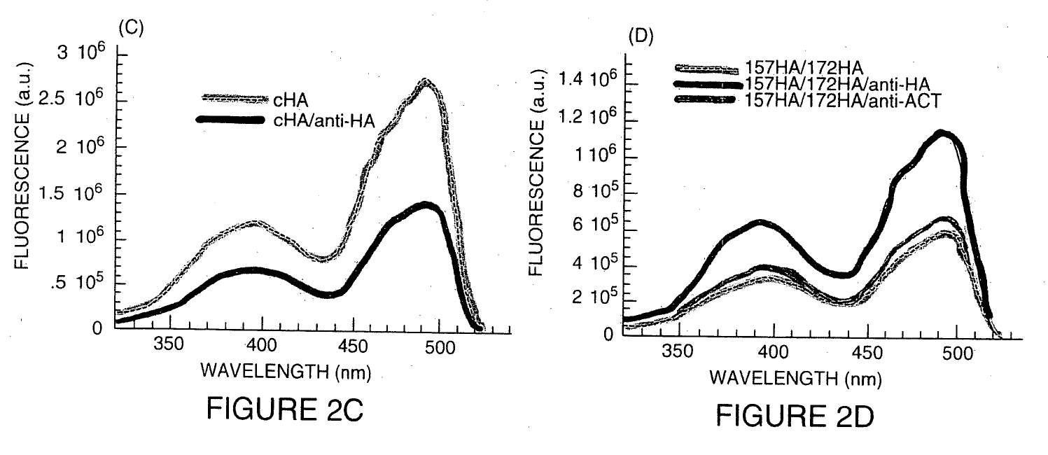

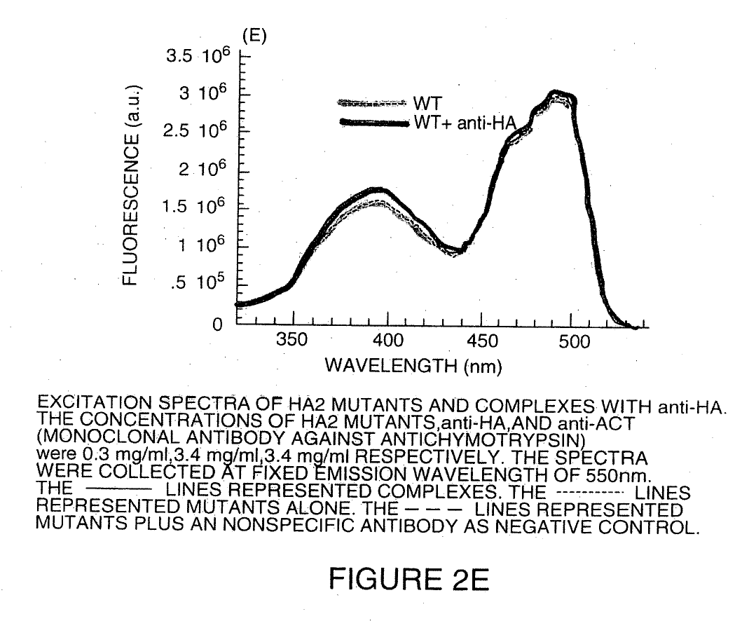

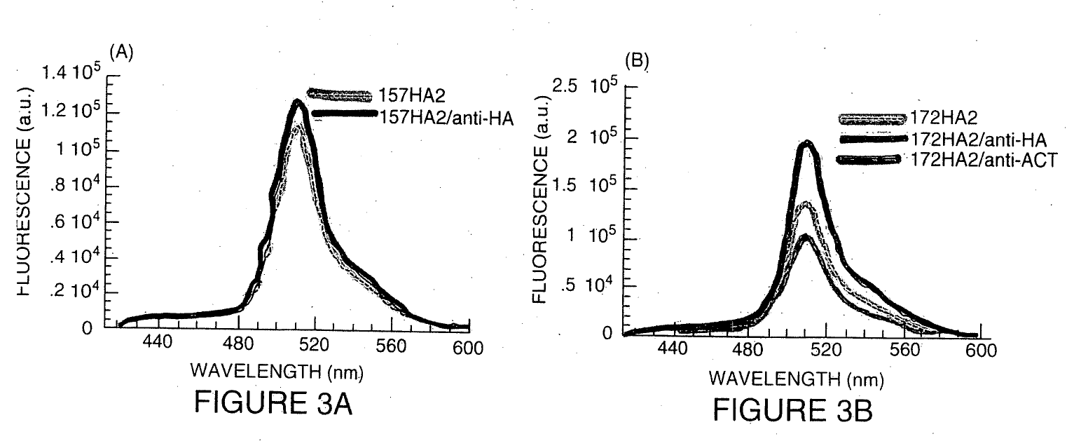

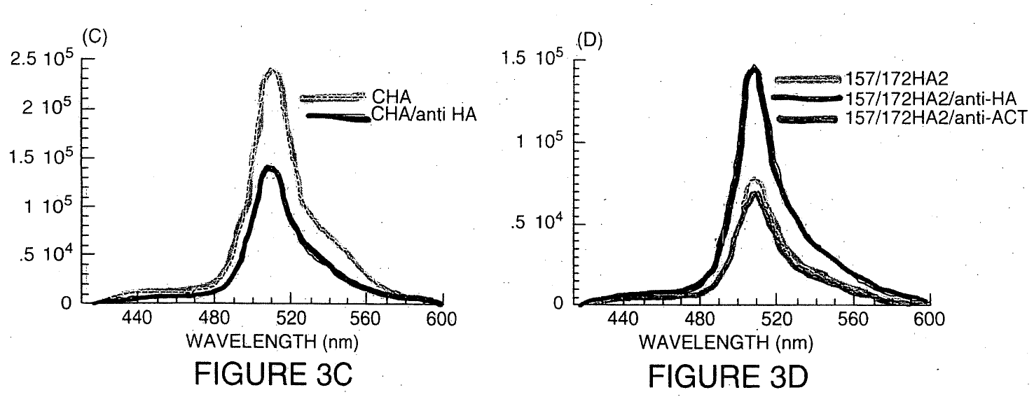

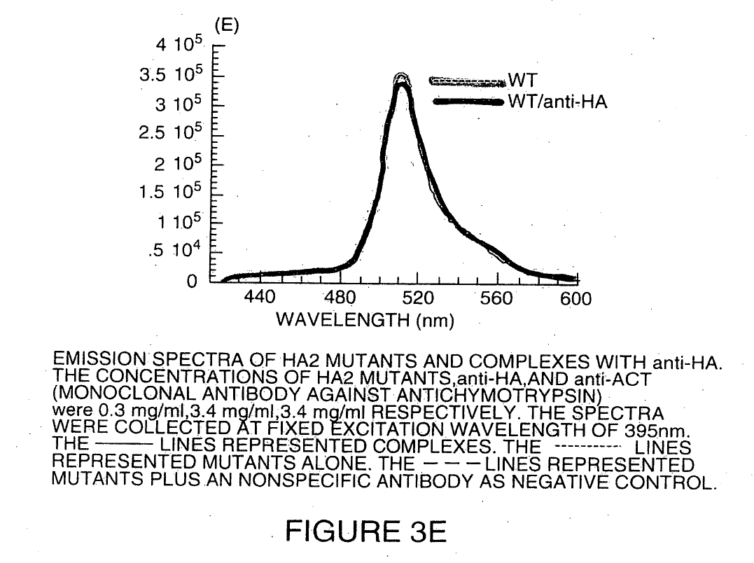

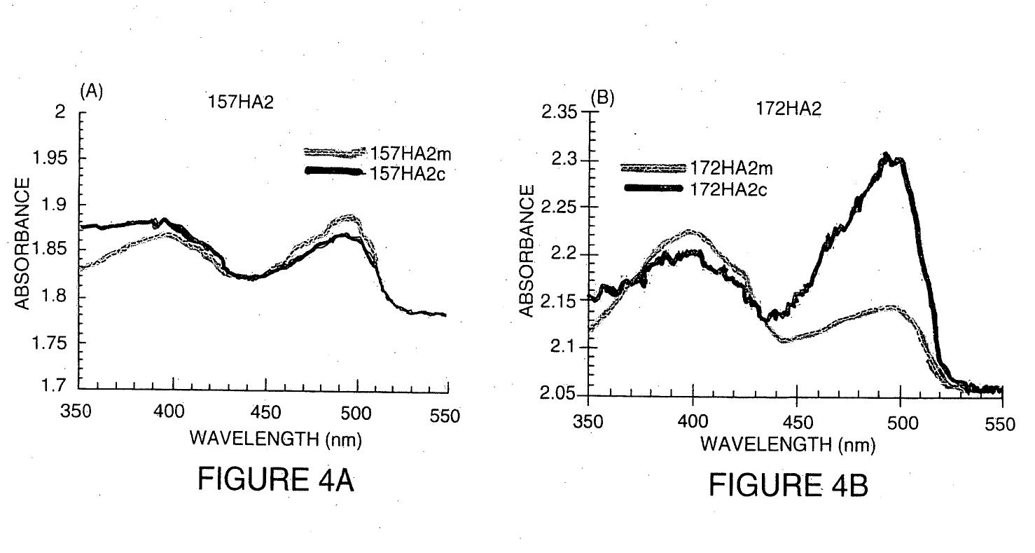

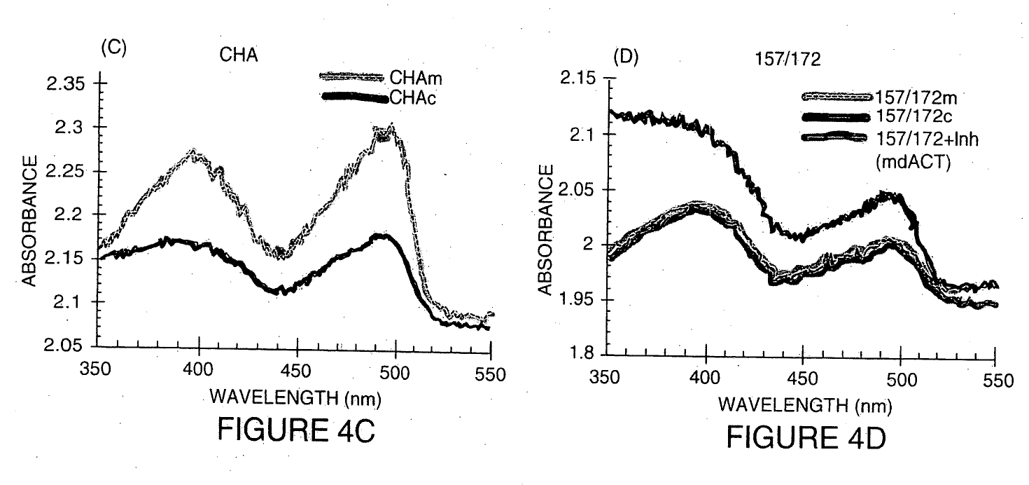

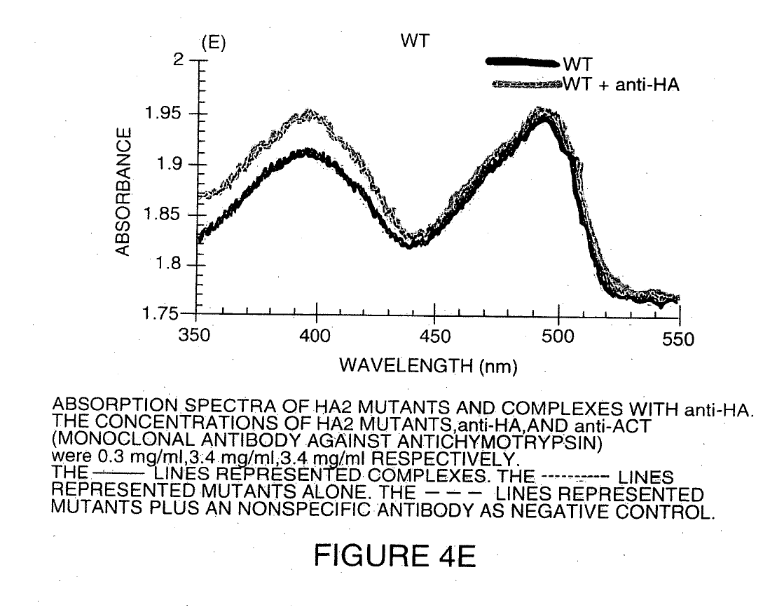

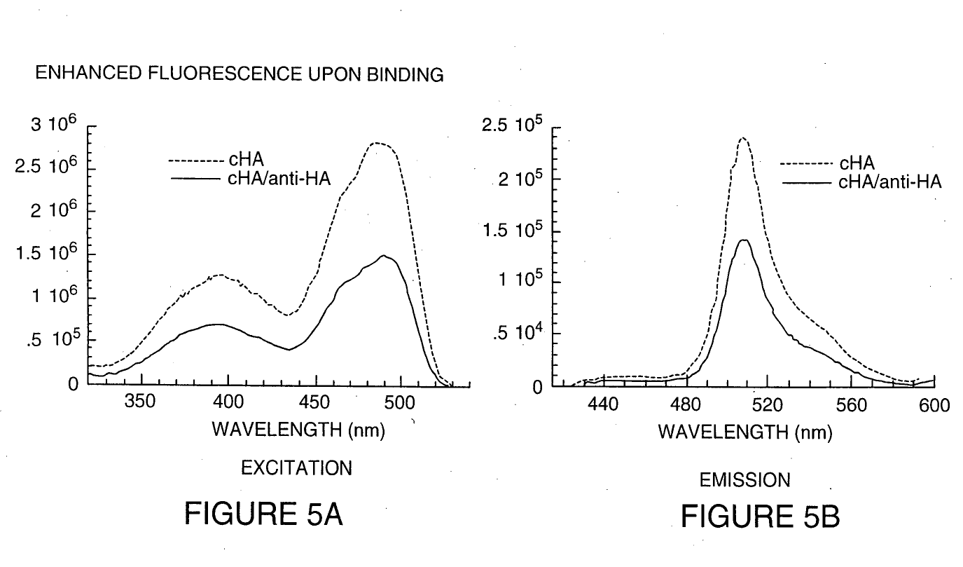

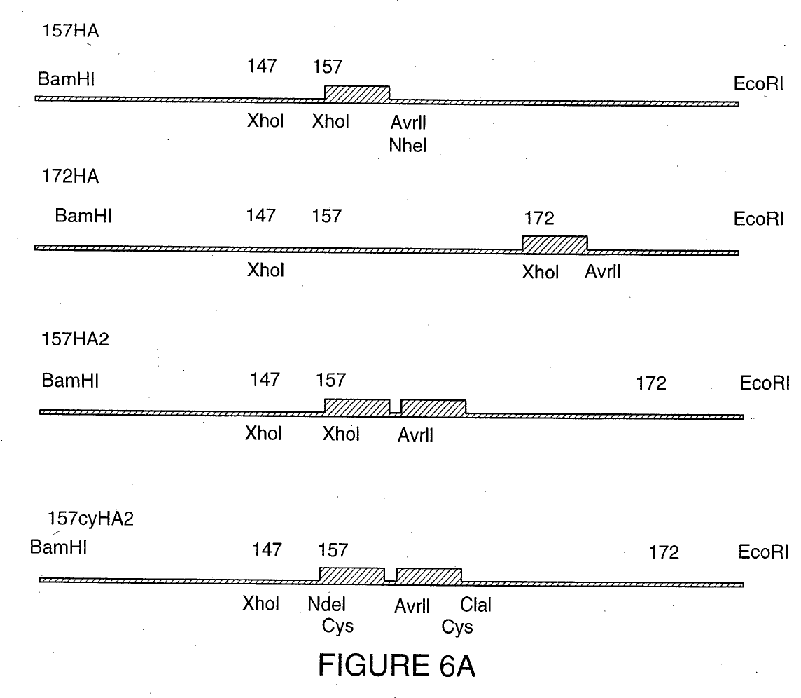

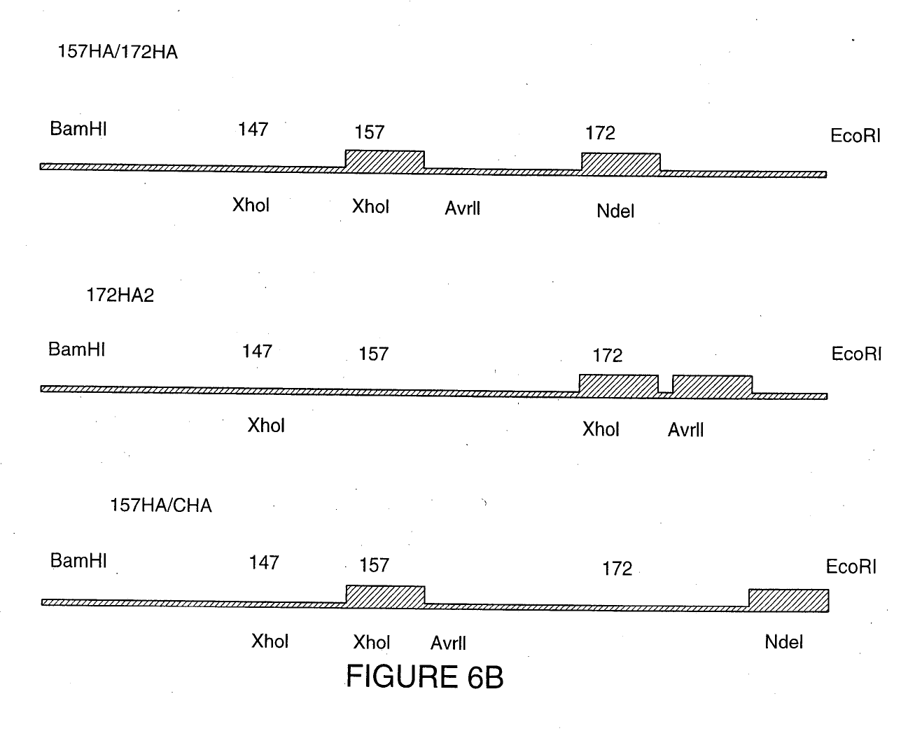

The following example demonstrates the production of a ligand activated affinity fluorescence protein (aFP) wherein the modified GFP molecule displays an altered spectra property when the binding site is engaged with (e.g., bound to) ligand relative to the spectral properties displayed when the binding site is unoccupied. This demonstration was dependent upon the development of assay conditions under which all the HA2 aFPs were complexed with anti-HA. The use of native electrophoretic analysis permits us to compare the absorption, excitation, and emission spectra between free and complexed (e.g., engaged with ligand) aFPs (FIG. 2A-2E, 3A-3E and 4A-4E). All four HA2 mutants (157HA2, 172HA2, CHA, 157HA/172HA) showed altered profiles (e.g., amplitude or spectral shape) in excitation, emission and absorption spectra after they formed complexes with anti-HA antibody. The absorption peak at 495 nm and fluorescence peak at 512 nm of complexed 157HA2 were slightly increased (FIGS. 2A, 3A, and 4A) while the fluorescence peak at 495 nm on excitation spectra remained unchanged. The absorption and fluorescence of complexed CHA decreased (FIGS. 2C, 3C, and 4C). Upon binding to the antibody, the 172HA2 mutant showed dramatic change in absorption, excitation, and emission. The excitation peak at 495 nm, emission peak at 512 nm and absorption peak at 495 nm, were significantly enhanced (FIGS. 2B, 3B, and 4B). The fluorescence intensity at 495 nm of excitation spectrum for complexed 172HA2 was about three fold higher than free 172HA2 while fluorescence intensity at 395 nm of excitation spectrum was about two times higher than free 172HA2 (FIG. 2B). The fluorescence and absorption of complexed 157HA/172HA are also enhanced (FIGS. 2D, 3D, and 4D). In fact, the fluorescence intensity at 512 nm for complexed 157HA/172HA was about three times higher than the one for free 157HA/172HA (FIG. 3D). The enhanced absorption and fluorescence properties appear to be specific to the HA tag insertion at 172 position since the excitation and emission spectra remained the same when nonspecific antibody was added to either 172HA2 or 157HA/172HA mutant. In addition, adding anti-HA antibody to wild-type GFP doesn't change the excitation, emission, and absorption spectra (FIGS. 2E, 3E, and 4E). In summary, this example demonstrates affinity fluorescent proteins (aFPs) which show different fluorescence properties upon the interaction of the binding site with its cognate ligand.

To enhance the difference of fluorescence signals between the free and bound forms of ligand-activated aFPs, the signal of a free aFP, which is similar to 172HA2 mutant without serine to proline point mutation at position 147, was reduced. Another mutation, S147P, was engineered in all the HA2 mutants to facilitate cyclization of the side chain and chromophore folding. Using crude cell lysate of EGFP172HA2, it was found that the fluorescence intensity at 495 nm of the excitation spectrum after adding the anti-HA antibody was about six times higher.

### EXAMPLE 2

### Ligand-Activated Affinity Fluorescent Protein Sensors Based on the Green Fluorescent Protein

Experimental Protocol

Expression Vectors. The original plasmid pEGFP purchased from Clontech (accession #U76561). Vector pProEX Hta from Life Technologies (cat.#10711-018) containing (His)6 tag at the amino-terminus for affinity purification. Restriction enzymes and DNA ligases were purchased from New England Biolabs (Beverly, Mass.). PCRs were performed on RoboCycler Gradient 96 (Stratagene) using PCR Supermix (Life Technologies). DNA purification and gel extraction were done using QLAGEN kits. GFP Constructs. EGFP was pulled out from pEGFP by BamHI/EcoRI digestion and cloned into MCS of pProEX HTa. All GFP constructs were made in pProEX HTa vector. GFPS147P was constructed using “megaprimer method” (G. Sarkar, S. Sommer BioTechniques Vol. 8, No. 4). Mutagenic primer containing substitution of Ser147 to Pro also carried XhoI site introduced by silent mutagenesis. EGFP157HA was done by PCR using primers 5′-GACAAGCAGCTCGAGTACCCCTACGACGTGCCCGACTACGCCCCTAGGGC TAGC-3′ (SEQ ID NO: 3) and 5′-GCCTCGAGACTGCAGGCTC-3′ (SEQ ID NO: 4). PCR product was digested by XhoI/EcoRI and inserted after Gln157 into Hta/EGFP. EGFP172HA was constructed the same way using primers 5′-ACATCGAGCTCGAGTACCCCTACGACGTGCCCGACTACGCCCCTAGGGAC GGCAG-3′ (SEQ ID NO: 5) and 5′-GCCTCGAGACTGCAGGCTC-3′ (SEQ ID NO: 6). After XhoI/EcoRI digestion PCR product was inserted after Glu172. For GFP157HA2, the second HA tag was amplified by PCR using primers 5′-GGGGGCCTAGGTACCCCTACGACGTGCCCGACTACGCCAAGAACGGCAT CAAGG-3′ (SEQ ID NO: 7) and 5′-GCCTCGAGACTGCAGGCTC-3′ (SEQ ID NO: 8). PCR product was digested by AvrII/EcoRI and ligated to GFP157HA. GFP172HA2 was constructed the same way as GFP157HA2 using primer 5′-GGGGGCCTAGGTACCCCTACGACGTGCCCGACTACGCCGACGGCAGCGT GCAGCTCGCC-3′ (SEQ ID NO: 9). GFP157HA/172HA was done by inserting NdeI restriction site into GFP157HA at the position Glu172 by “megaprimer method”. HA tag was amplified using primers 5′-GGGGGCATATGTACCCCTACGACGTGCCCGACTACGCCGACGGCAGCGTG CAG-3′ (SEQ ID NO: 10) and 5′-GCCTCGAGACTGCAGGCTC-3′ (SEQ ID NO: 11). PCR product was digested by NdeI/EcoRI and ligated to GFP157HA/172NdeI. GFP157HA/CHA was generated using GFP157HA as a template by insertion of HA tag at C-terminus before “stop” codon. HA tag was amplified by PCR with primers: 5′-GAGCTGTACAAGCATATGTACCCCTACGACGTGCCCGACTACGCCTAAAG CGGCCGCGAC-3′ (SEQ ID NO: 12) and 5′-GCCTCGAGACTGCAGGCTC-3′ (SEQ ID NO: 13).

Expression and Purification of HA Mutants. All GFP constructs were expressed in E. coli BL21 with IPTG induction. Transformed cells were grown at 37° C. to an OD600 of 0.6-0.8, then was induced by adding isopropyl-1-thio-b-D-galactopyranoside to 0.2 mM. After incubation at 30° C. for about 18 hours, cells were transferred to 4° C. Cells were harvested after staying at 4° C. for 48 hours. HA mutants were purified using His6 affinity chromatography, nickel-NTA Superflow column (QLAGEN). The proteins were about more than 90% pure verified by SDS-PAGE. The concentrations of GFP and mutants were determined by Bradford method (BioRad, Richmond, Calif.).

Formation of HA2-Anti-HA Complexes. The complexes used for nondenaturing PAGE analysis and absorption spectra were formed by incubating 0.3 mg/ml (μM) of HA2 mutants and 3.2 mg/ml anti-HA in PBS buffer at 23° C. for an hour, then 4° C. for 18 hours. The complexes used for excitation and emission spectra were formed by incubating 0.025 mg/ml (μM) of HA2 mutants and 0.20 mg/ml anti-HA in PBS buffer at 23° C. for an hour, then at 4° C. for 18 hours.

Absorption, Excitation, and Emission Spectra. The absorption spectra were collected on an AVIV Model 118DS spectrophotometer. (AVIV Associates, Inc., Lakewood, N.J.) at 25° C. Excitation and emission spectra were recorded on a Fluorolog 3-22 spectrofluorimeter (Instruments S.A., Inc., Edison, N.J.) at 25° C. The instrument parameters are the following: slit of 2.5 nm, integration time of 0.5 second, interval of 1 nm, and PMT 950V.

Binding Assay on Solid Surface. Three identical dots for each mutant containing 1 μl of 0.3 mg/ml were spotted on the nitrocellulose membrane and air dry for 5 minutes at 23° C. Then 1 μl of PBS buffer, 2.2 mg/ml of anti-HA, and 2.2 mg/ml BSA were spotted on the second and third column, respectively. The membrane was incubated or at 23° C. for 5 minutes and was washed in PBS buffer with 0.5% Tween 20, pH 7.4 on a shaker at 23° C. for 15 minutes. The membrane was photographed on an UV lamp excited with 365 nm UV light.

Emission Spectra of aFP 172HA2 Alone and aFP 172HA2/anti-HA Complex on the Nitrocellulose Membrane. The spots having 172HA2 alone, aFP172HA2/anti-HA complex, and 172HA2 with BSA were cut from the nitrocellulose membrane respectively, attached to a glass cover slip by moisture respectively. The width of the cover slide was snug in the diagonal of 1 cm quartz cuvette so that the excitation light and emission light were 45 degrees to the glass cover slip, respectively. The mission spectra were recorded on a Fluorolog 3-22 spectrofluorimeter at 25° C. The instrument parameters are the following: slit of 4 nm, integration time of 1 second, interval of 1 nm, and PMT of 950V.

Results

S147P Stabilized the Fluorescence of HA Mutants.

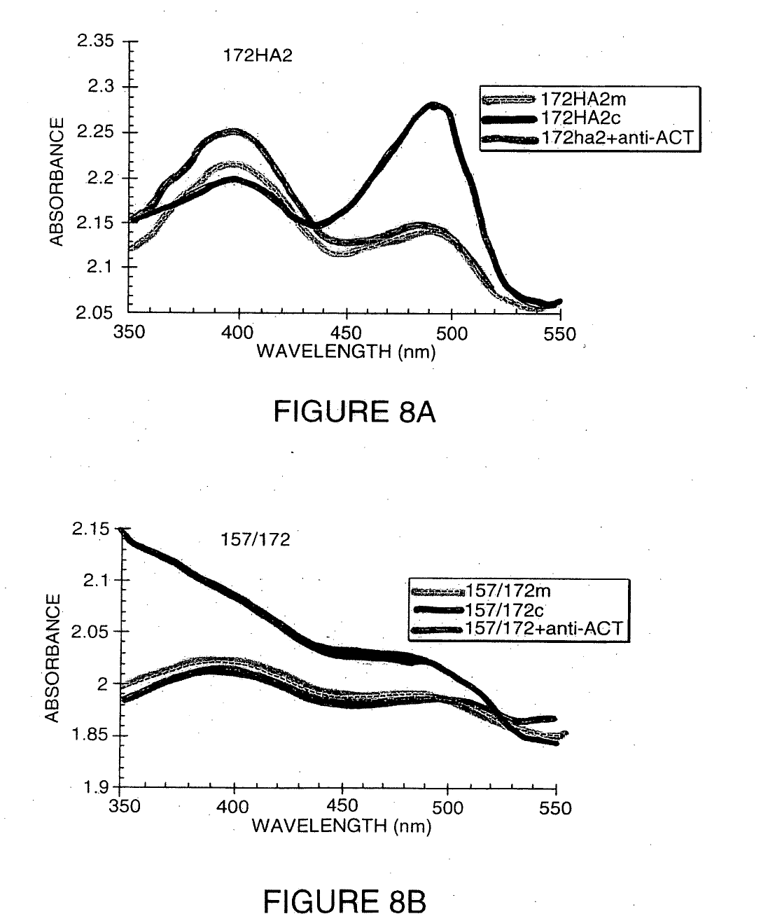

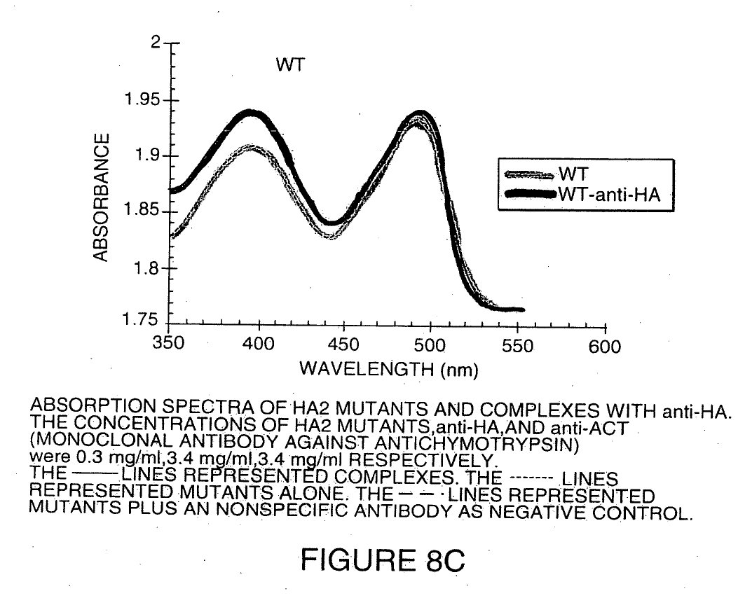

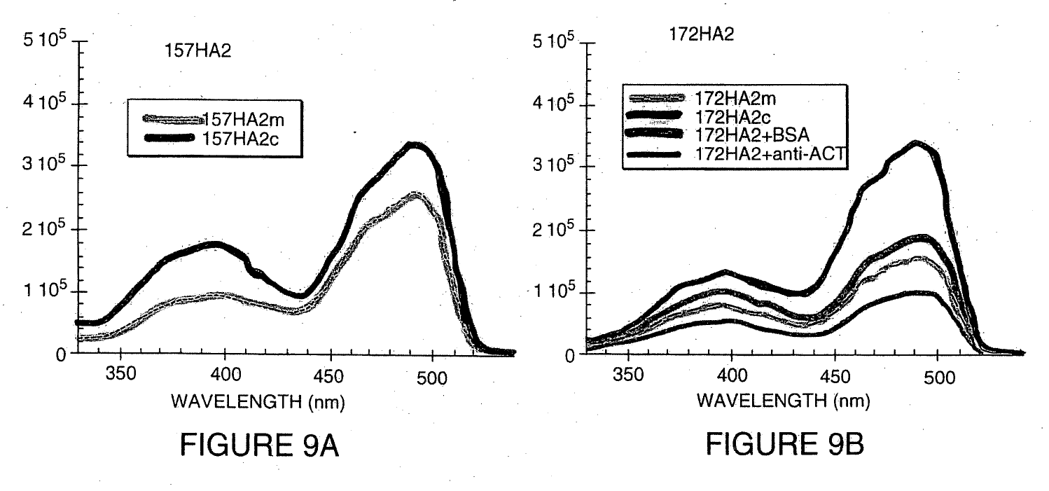

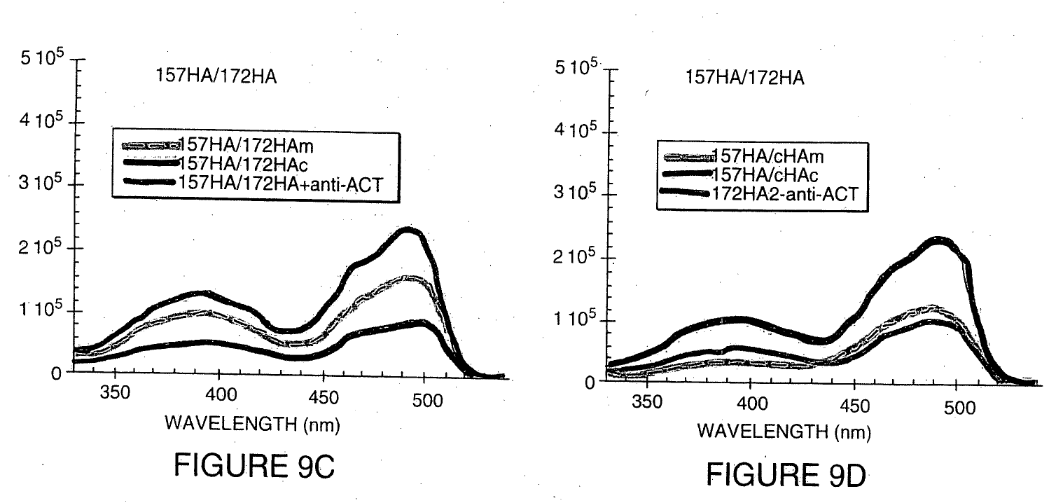

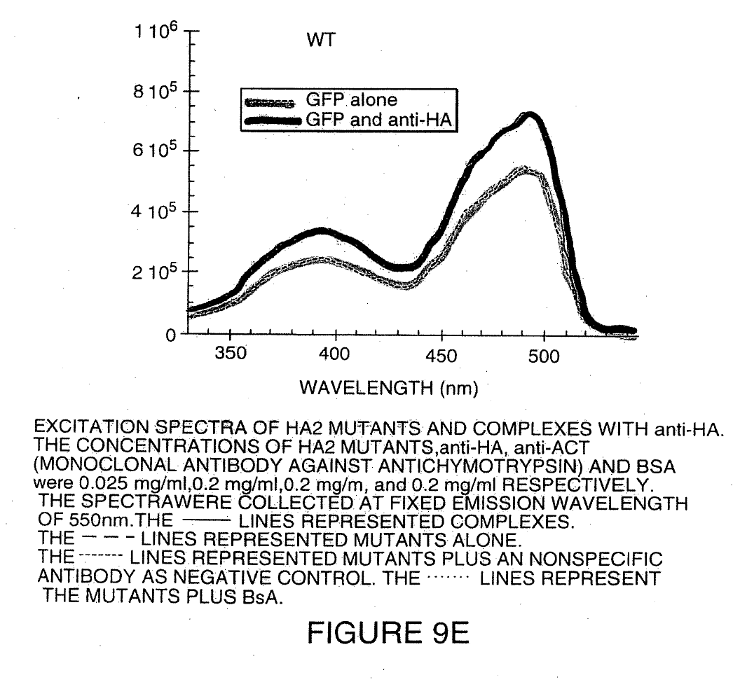

An epitope from haemagglutinin (HA tag contains YPYDVPDYA (SEQ ID NO: 2) that is recognized by the monoclonal antibody, 12CA5 or anti-HA) was inserted into the locations of Gln157-Lys158, Glu172-Asp173 and c-terminal of the GFP (Table). Since the fluorescence of the HA mutants was not stable, serine at position 147 was mutated to proline to stabilize the fluorescence. Therefore, all the mutants had S147P point mutation. EGFP/S147P was the wild type protein as control and was referred as GFP for brevity. EGFP has a single absorption and excitation peak at 495 nm because of S65T mutation. However, GFP with the S147P mutation gave two excitation peaks centering at 395 nm and 495 nm as well as two absorption peaks centering at 395 nm and 495 nm (FIGS. 8C and 9E). The excitation peak at 495 nm was about twice as high as the one at 395 nm. The S147P mutation likely shifts the equilibrium of the chromophore toward the neutral or protonate form. All the mutants listed in the Table have two absorption and excitation peaks similar to EGFP/S147P due to the S147P mutation.

HA2 Mutants formed Specific Fluorescent Complexes with the Anti-HA Antibody.

GFP157HA and GFP172HA were fluorescent and able to form immunocomplexes with anti-HA antibody as was demonstrated by immunoprecipitation using the anti-HA antibody. This result showed that 157HA and 172HA were pulled down specifically by the anti-HA antibody. However, the complexes couldn't be detected on nondenaturing PAGE suggesting that the HA mutants were bound to anti-HA with low affinity. To improve the binding, two tandem HA tags were inserted at either Gln157-Lys158 (157HA2) or Glu172-Asp173 (172HA2) (Table). 157HA/172HA contains one HA tag between Gln157 and Lys158 and another HA tag between Glu172 and Asp173. 157HA/cHA contains one HA tag between Gln157 and Lys158 and another HA tag at the C-terminal of the GFP. The mutants having two HA tags were designed as HA2 mutants. All the HA2 mutants formed specific fluorescent complex with anti-HA. In contrast, no fluorescent complex band was observed for the sample containing wt-GFP and anti-HA or the sample containing 157HA/172HA with non-specific antibody as they migrated at the similar speed as 172HA2 and 157HA/172HA alone. The association constants between HA2 mutants and anti-HA measured using dot blot assay on the nitrocellulose membrane were at nanomolar range indicating strong binding between the HA2 mutants and the anti-HA antibody.

Comparison of Absorption, Excitation, and Emission Profiles for HA2 Alone and HA2/Anti-HA Complexes.

The HA2 mutants were fully bound to the anti-HA and no free HA2 mutants was left in solution. This allowed measurement and comparison of the spectra of absorption, excitation, and emission for the complexes with ones of mutants alone quantitatively (Figures. 8A-8C, 9A-9E, and 10A-10E). The absorption peak at 495 nm for the complexed 172HA2 was increased about 1.5 times compared with 172HA2 alone, or with 172HA2 in the presence of non-specific antibody, while the peak at 395 nm was decreased a little (FIG. 8A). Both absorption peaks for complexed 157HA/172HA were increased 2 times compared with 157HA/172HA alone. The peaks remained unchanged for the negative control sample having 157HA/1172HA and nonspecific antibody (FIG. 8B). The absorption spectrum for the sample of wild type GFP plus the anti-HA was similar to the one of wild type GFP alone (FIG. 8C). The data indicated that the absorption peak change at 495 nm reflected the specific binding between HA2 mutants and the anti-HA antibody.

All HA2 mutants showed different levels of fluorescence enhancement upon binding of anti-HA antibodies. 157HA2 and 157/cHA mutants formed aggregates with the anti-HA if the concentration of these proteins in the reaction solution was above 0.3 mg/ml. The concentration was 3.2 mg/ml for the anti-HA respectively. The excitation and emission spectra were taken at the concentration of 0.025 mg/ml and 0.20 mg/ml for HA2 mutants and the anti-HA, respectively. The same concentration (0.20 mg/ml) for non-specific antibody or BSA as negative control was used.

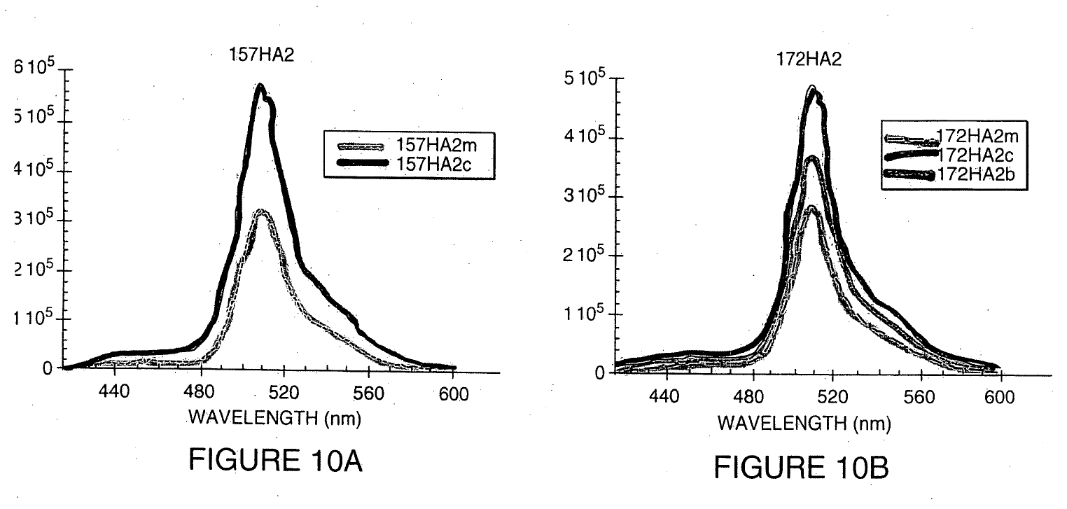

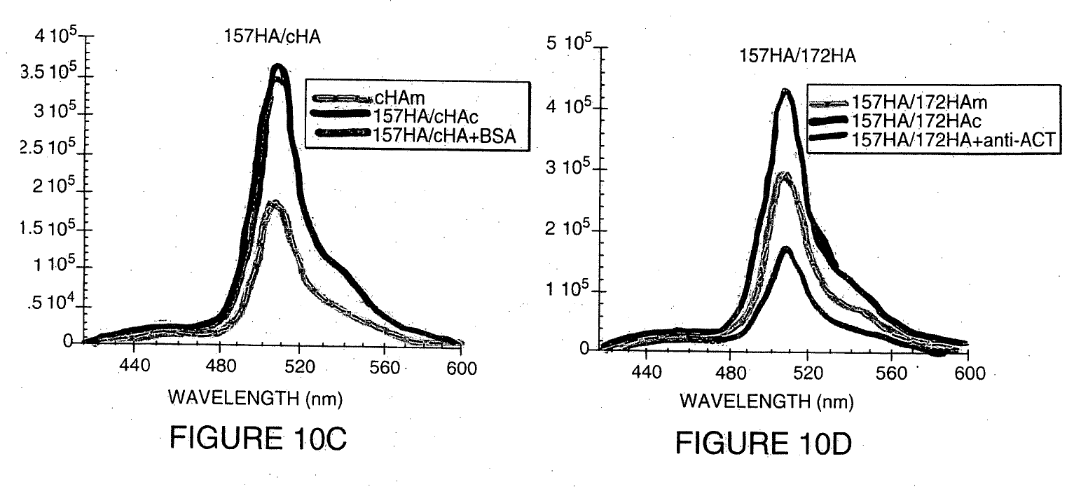

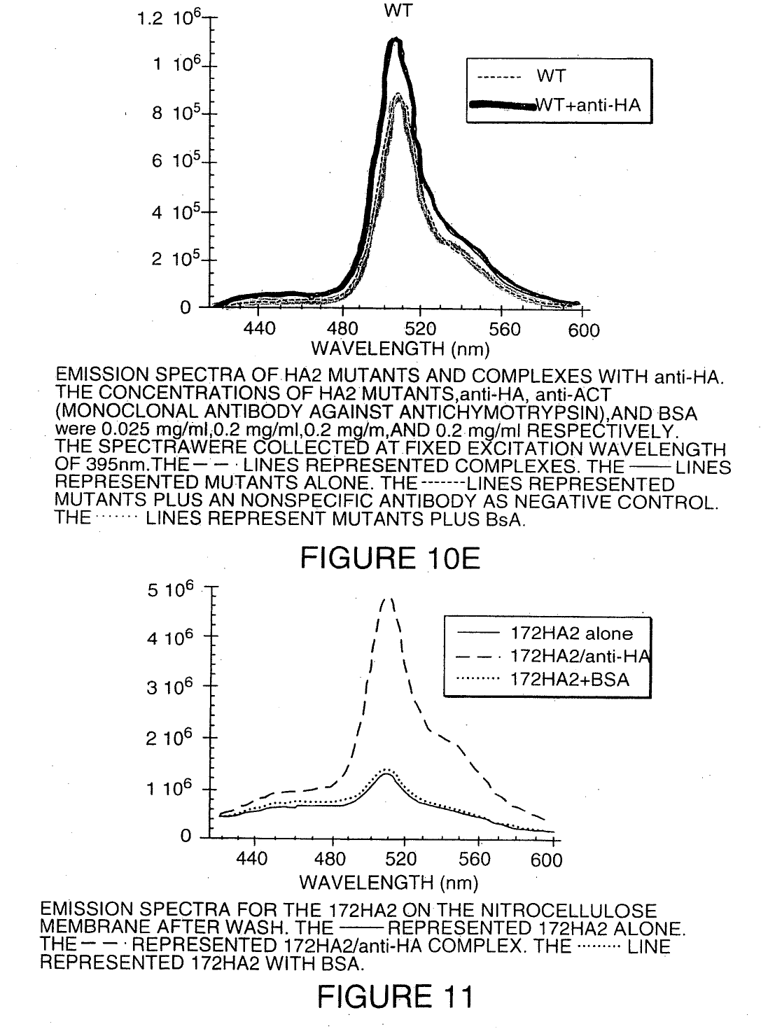

The excitation peaks at 395 nm and 495 nm were increased, ranging from one- to three-fold for four HA2 mutants once they were in the complexes with the anti-HA antibody (FIGS. 9A-9E). The largest enhancement of the fluorescence at 495 nm was given by the 172HA2-anti-HA complex. The fluorescence intensity at 495 nm of excitation spectrum for complexed 172HA2 was about three times higher than 172HA2 alone while fluorescence intensity at 395 nm of excitation spectrum was about two times higher than free 172HA2 (FIG. 9B). The emission spectra also showed that the fluorescence intensity at 510 nm was enhanced from one- to three-fold for all the HA2 mutants upon binding to the anti-HA (FIGS. 10A-10E). Adding nonspecific antibody to 157HA/172HA didn't result in any enhancement of the fluorescence (FIGS. 9D and 10D). In the contrast, the fluorescence was decreased (FIGS. 9D and 10D).

The excitation and emission spectra for the wild type GFP were also increased in the presence of the anti-HA (FIGS. 9E and 10E). This result indicated that the enhancement of the fluorescence likely resulted either from the specific binding between HA2 mutants and the anti-HA antibody or from the nonspecific binding between wild type GFP and the anti-HA antibody. When bovine serum albumin was added to the 172HA2 or 157HA/cHA mutants in the solution, the fluorescence intensity of the excitation peaks at 395 nm and 495 m was increased, so was the fluorescence intensity of the emission peak at 510 nm (FIGS. 9B and 9C, FIGS. 10B and 10C). In the case of 157HA/cHA mutant, the fluorescence intensity of the excitation peak at 495 nm in the presence of BSA was even higher than the one in the presence of the anti-HA (FIG. 9B). In addition, the intensity of the emission peak at 510 nm for the sampling containing 157HA/cHA and BSA was almost identical to the one for the sample containing 157HA/cHA and anti-HA. The interference of the nonspecific binding proteins made it difficult to distinguish whether the enhanced fluorescence was because of the specific interactions or nonspecific interactions.

Binding Assay on Solid Surface.

To avoid the problem caused by nonspecific interactions of proteins in solution, a rapid and simple binding assay on solid phase support was developed. Under 365 nm UV light, the second column labeled αaHA and the third one labeled +BSA gave much brighter fluorescence signals than the one labeled alone before wash. After the membrane was washed in PBS buffer with 0.5% Tween-20 for 15 minutes, only the second column labeled αaHA remained brighter fluorescence signal while the one labeled alone and the one labeled +BSA gave very dim fluorescence. The fluorescence intensity for three dots for wild type GFP was similar. To test whether the reduced fluorescence signal was due to loss of protein during the washing step, the amount of the protein for the membrane after wash using the antibody against the GFP by western blot was quantified. It was found that three dots for each mutant had similar amount of protein. Therefore, the enhanced fluorescence signal resulted from the specific binding between the mutants and the anti-HA. This result demonstrated that all the mutants attached on solid support are able to bind the target selectively. The binding can be reported based on the enhancement of the fluorescence.

Most strikingly, the solid surface assay can detect the weak binding that cannot be detected in solution. For example, the binding between 157HA and anti-HA or the one between 172HA and anti-HA, which was at micromolar range, was detected only by immunoprecipitation, but not by nondenaturing PAGE gel. However, the weak binding was captured on the nitrocellulose membrane. This result clearly showed that the binding assay on the solid surface is more sensitive and specific than the one in solution. The washing step not only eliminates the non-specific binding, but also increases the signal to noise ratio. As shown in FIG. 11, the signal to noise ratio was increased to 28 fold if we normalized the difference between the maximum intensity of 172HA2/anti-HA complex and 172HA2 alone at 510 nm versus the difference between the maximum intensity of 172HA2 with BSA and 172HA2 alone at 510 nm. It is worth mentioning that the GFP and mutants spotted on the nitrocellulose membranes were still fluorescent after storage for a month at 4 degrees.

TABLE

| Mutants | Mutation | Insertion Site | Fluorescence |
|---|---|---|---|
| 157HA | Ser147Pro | | Yes |
| | One HA insertion | Gln157-Lys158 | |
| 172HA | Ser147Pro | | Yes |
| | One HA insertion | Glu172-Asp173 | |
| 157HA2 | Ser147Pro | | Yes |
| | Two HA insertion | Glu172-Asp158 | |
| 172HA2 | Ser147Pro | | Yes |
| | Two HA insertion | Glu172-Asp173 | |
| 157HA/172HA | Ser147Pro | | Yes |
| | One HA insertion | Gln157-Lys158 | |
| | One HA insertion | Glu172-Asp172 | |
| 157HA/cHA | Ser147Pro | | Yes |
| | One HA insertion | Gln157-Lys158 | |
| | One HA insertion | At C-terminal | |

Note:
1) All the mutants have a mutation from serine to proline at the position 147.
2) The mutant 157HA2 and 172HA2 are composed of two HA tags in tandem. The mutant 157HA/172HA has a HA tag inserted in between Gln 157 and Lys158 and another HA tag inserted in between Glu172 and Asp173. The mutant 157HA/c-HA has a HA tag inserted in between Gln157 and Lys158 and another HA tag fused at the C-terminal.

Discussion
Monomeric GFP Biosensors.

Four kinds of biosensors derived from the monomeric GFP including our aFPs, such as pH sensors, calcium sensor, and BLIP sensor were reported up to date ( ). The detection of these GFP sensors all depend on the enhancement of fluorescence intensity. Recently, Richmond et al. engineered two mutants (10C-S147H/Q204H/S202D and 10C-S147H/Q204H/F223E) of GFP, in which copper was chelated by residue His 147 and His204. The fluorescence of the two mutants was quenched about 80% at about 100 μM copper. However, the third type of GFP biosensor illustrated in FIG. 7 based on wavelength shifted fluorescence once they bind to the targets is not available yet.

The reason why an increased fluorescence signal upon binding to protein targets remains unclear. The simple interpretation could be that the environment of the chromophore may become more hydrophobic and be more protected from quenching upon binding to target proteins. The atomic structure of an aFP/anti-HA complex will shed light on making new biosensors. Up to date, all the wavelength shift mutants of GFP are due to the mutations either in the chromophore or in the vicinity of the chromophore. The structure analysis of yellow fluorescent protein (YFP) showed that the T203Y mutation is responsible for the long wavelength shift from 508 nm to 527 nm. The aromatic ring of the tyrosine at 203 position is stacking on the phenolate anion of the chromophore to add additional polarizability around the chromophore. Recently, a fluorescent protein emitting red fluorescence at 583 nm, isolated from Anthozoa species was cloned and expressed. Matz et al. postulated that 4 tryptophans residues, two of which (positions 94 and 145) located near the chromophore may absorb UV light and then transfer to the chromophore that emits long wavelength light (Matz, M. V., et al., Nat. Biotechnol., 17(10):969-973 (1999)). Matz et al. also thought that an additional autocatalytic reaction may lead to a more extended conjugated π-system. Therefore, wavelength shift aFP sensors will likely be generated by introducing tryptophan residues into the molecular recognition sites and the immediate vicinity of the chromophore of aFPs. Upon binding to the target, aFP may emit long wavelength by absorbing the energy produced by the fluorescence energy transfer occurring between tryptophan residues at the molecular recognition site and the ones in the immediate vicinity of the chromophore.

Generation of aFP Biosensor with High Affinity.

The 157 and 172 locations appeared to be robust for introducing various foreign peptide sequences. 5 different peptide binding sequences were inserted into 157 and 172 locations, respectively. It was found that most of mutants with insertion are fluorescent. However, it is challenging to have high affinity binding GFP mutants to the targets. The affinity of HA mutants to the anti-HA was improved by using two tandem HA tags. It is interesting to point out that the GFP 157HA/172HA and GFP 157HA/CHA bind to the anti-HA more tightly than GFP 157HA, or GFP 172HA. Adding HA at other site inserted in 157, 172 and C-terminal location, respectively can improve the binding about 1000 fold. The higher affinity may be either due to the different conformation or the stoichiometry ratio between the HA mutants and the anti-HA.

Serine protease inhibitors were generated by inserting a peptide reactive loop at 157 and 172 locations that may inhibit serine proteases, such as chymotrypsin and trypsin. By randomizing P2, P1 and P2′ position, we generated two small libraries. The two cysteines flanking at P3 and P6 may form disulfide bond so that a cyclic peptide may be formed and sits on the top of the surface loop. One fluorescent mutant isolated from the library at the 157 location and 4 fluorescent mutants isolated from the library at the 172 location were able to form complexes with trypsin-agarose beads. However, no fluorescent complex was shown on nondenaturing gel due to low inhibitory activity. This result which is in line with the result of HA insertion mutants demonstrated that the position 157 and 172 are fluorescence insensitive sites and suitable for introducing foreign peptide binding sequences.

Binding Assay on Solid Surface (Prototype of Protein Chip). To use monomeric GFP based biosensors in real applications, the detection limit in solution requires the affinity constant in nanomolar range (FIGS. 9A-9E and 10A-10E). However, the detection sensitivity is increased tremendously using solid surface assay after immobilizing aFPs on nitrocellulose membrane. The solid surface assay is able to detect the weak binding in μM range that cannot be detected in solution. The solid surface assay is also capable to detect protein-protein binding in more complex environment since the nonspecific binding will be eliminated by the washing step. It should be noted that the absorbed GFP on the nitrocellulose membrane was still fluorescent after storage at 4 degrees for a month.

While this invention has been particularly shown and described with references to preferred embodiments thereof, it will be understood by those skilled in the art that various changes in form and details may be made therein without departing from the scope of the invention encompassed by the appended claims.

What is claimed is:

1. An affinity fluorescent protein comprising a modified flourescent protein molecule which comprises a mutated flourescent protein molecule and a heterologous amino acid sequence, thereby introducing a ligand-activated protein binding site, wherein the modified flourescent protein molecule displays an altered spectral property when the binding site is engaged with ligand relative to the spectral property displayed when the binding site is not engaged by ligand.

2. An affinity fluorescent protein comprising a modified GFP molecule which comprises a mutated GFP molecule and a heterologous amino acid sequence, thereby introducing a ligand-activated protein binding site, wherein the modified GFP molecule displays an altered spectral property when the binding site is engaged with ligand relative to the spectral property displayed when the binding site is not engaged by ligand.

3. The affinity fluorescent protein of claim 2 wherein the mutated GFP comprises a substitution of Ser147Pro.

4. The affinity fluorescent protein of claim 2 wherein the altered spectral property is selected from the group consisting of: an altered absorption spectra, an altered excitation spectra, an altered emission spectra and any combination thereof.

5. The affinity fluorescent protein of claim 4 wherein the modified GFP molecule comprises one or more protein binding sites introduced at a single site in tandem or introduced at distinct sites as separate heterologous sequences.

6. The affinity fluorescent protein of claim 5 wherein the modified GFP molecule comprises protein binding sites introduced into a loop present on the surface of the GFP molecule and the presence of the heterologous amino acid sequences does not alter the spectral properties of the GFP.

7. The affinity fluorescent protein of claim 6 wherein the modified GFP molecule comprises at least one heterologous amino acid sequence introduced at a location of the GFP molecule selected from the group consisting of: the N-terminus, between Gln157 and Lys158, between positions Glu172 and Asp173, the C-terminus and any combination thereof.

8. The affinity fluorescent protein of claim 7 wherein the mutated GFP comprises a substitution of Ser147Pro.

9. The affinity fluorescent protein of claim 8 wherein the affinity fluorescent protein comprises at least one protein binding site comprising a heterologous amino acid sequence introduced between Gln157 and Lys158 of the GFP molecule.

10. The affinity fluorescent protein of claim 8 wherein the affinity fluorescent protein comprises at least one protein binding site comprising a heterologous amino acid sequence introduced between Glu172 and Asp173 of the GFP molecule.

11. An affinity fluorescent protein expression cassette comprising a modified GFP nucleic acid sequence which is mutated and operatively linked to expression control sequences, wherein the modified GFP sequence comprises a recombinant peptide which comprises restriction endonuclease sites introduced at a location of the GFP molecule selected from the group consisting of: between Gln157 and Lys158, between Glu172 and Asp173 and both of the aforementioned locations.

12. The affinity fluorescent protein expression cassette of claim 11, wherein the recombinant peptide comprises the hexapeptide LEPRAS.

13. The affinity fluorescent protein of claim 11 wherein the mutated GFP comprises a substitution of Ser147Pro.

14. An affinity fluorescent protein expression vector comprising a modified GFP nucleic acid sequence which is mutated and operatively linked to expression control sequences, wherein the modified GFP sequence comprises a heterologous amino acid sequence introduced at a position of the GFP molecule selected from the group consisting of: between Gln157 and Lys158, between Glu172 and Asp173 and both of the aforementioned locations.

15. The affinity fluorescent protein expression vector of claim 14 wherein the mutated GFP comprises a substitution of Ser147Pro.

16. A host cell, comprising:
a recombinant nucleic acid molecule which comprises expression control sequences operatively linked to a nucleotide sequence encoding an affinity fluorescent protein, wherein said affinity fluorescent protein comprises a modified GFP molecule which comprises a mutated GFP molecule and a heterologous amino acid sequence which functions as a ligand-activated protein binding site, wherein the affinity fluorescent protein displays an altered spectral property when the binding site is engaged with ligand relative to the spectral property displayed when the binding site is not engaged by ligand.

17. The host cell of claim 16 wherein the heterologous amino acid sequence is introduced at a location of the GFP molecule selected from the group consisting of: between Gln157 and Lys158, between Glu172 and Asp173 and both of the aforementioned locations.

18. The host cell of claim 16 wherein the mutated GFP comprises a substitution of Ser147Pro.

19. A method of detecting the presence of a target ligand in a mixture of macromolecules comprising the steps of:
(a) preparing a sample to be evaluated for the presence of a target ligand molecule;
(b) contacting the sample of (a) with an affinity fluorescent protein which comprises a binding site for the target ligand;
(c) exciting the affinity fluorescent protein with light;
(d) measuring the fluorescent property that differs as a result of ligand activation of the affinity fluorescent protein.

20. The method of claim 19, wherein the fluorescent property that differs as a result of ligand activation is selected from the group of properties consisting of: amplitude of the excitation, absorption or emission spectra and shape of the any of the aforementioned spectras.

21. The method of claim 19 wherein the aFP comprises a modified fluorescent protein or molecule, such as a modified GFP molecule, which comprises a mutated GFP molecule and a heterologous amino acid sequence, thereby introducing a ligand-activated protein binding site, wherein the modified fluorescent protein displays an altered spectral property when the binding site is engaged with ligand relative to the spectral property displayed when the binding site is not engaged by ligand.

22. The method of claim 19 wherein the fluorescent property is measured using a solid support phase.

23. The method of claim 22 wherein the solid support phase is selected from the group consisting of: nitrocellulose and a protein chip.

24. A method of detecting the occurrence of a target ligand in a cell comprising the steps of:
a) introducing into the cell an aFP which comprises a binding site for the target ligand;
(c) exciting the affinity fluorescent protein present in the cell with light;
(d) detecting a pattern of fluorescence due to ligand activation of the affinity fluorescent protein in the cell of (c) and comparing it to the pattern of fluorescence in a control cell,
wherein the pattern of fluorescence determines the occurrence of the target ligand in the cell.

25. The method of claim 24 wherein the aFP comprises a modified fluorescent protein or molecule, such as a modified GFP molecule, which comprises a mutated GFP molecule and a heterologous amino acid sequence, thereby introducing a ligand-activated protein binding site, wherein the modified fluorescent protein displays an altered spectral property when the binding site is engaged with ligand relative to the spectral property displayed when the binding site is not engaged by ligand.

26. The method of claim 24 wherein the cell is selected from the group consisting of: a macrophage and a yeast cell.


SEQUENCE LISTING (13 sequences)

SEQ ID NO: 1 - protein, 6 aa, Artificial Sequence
  Hexapeptide
LEPRAS

SEQ ID NO: 2 - protein, 9 aa, Artificial Sequence
  Haemagglutinin epitope
YPYDVPDYA

SEQ ID NO: 3 - DNA, 54 bp, Artificial Sequence
  primer
gacaagcagctcgagtacccctacgacgtgcccgactacgcccctagggctagc

SEQ ID NO: 4 - DNA, 19 bp, Artificial Sequence
  primer
gcctcgagactgcaggctc

SEQ ID NO: 5 - DNA, 55 bp, Artificial Sequence
  Primer
acatcgagctcgagtacccctacgacgtgcccgactacgcccctagggacggcag

SEQ ID NO: 6 - DNA, 19 bp, Artificial Sequence
  Primer
gcctcgagactgcaggctc

SEQ ID NO: 7 - DNA, 54 bp, Artificial Sequence
  Primer
gggggcctaggtacccctacgacgtgcccgactacgccaagaacggcatcaagg

SEQ ID NO: 8 - DNA, 19 bp, Artificial Sequence
  Primer
gcctcgagactgcaggctc

SEQ ID NO: 9 - DNA, 59 bp, Artificial Sequence
  Primer
gggggcctaggtacccctacgacgtgcccgactacgccgacggcagcgtgcagctcgcc

SEQ ID NO: 10 - DNA, 53 bp, Artificial Sequence
  primer
gggggcatatgtacccctacgacgtgcccgactacgccgacggcagcgtgcag

SEQ ID NO: 11 - DNA, 19 bp, Artificial Sequence
  Primer
gcctcgagactgcaggctc

SEQ ID NO: 12 - DNA, 60 bp, Artificial Sequence
  Primer
gagctgtacaagcatatgtacccctacgacgtgcccgactacgcctaaagcggccgcgac

SEQ ID NO: 13 - DNA, 19 bp, Artificial Sequence
  primer
gcctcgagactgcaggctc